(19) United States
(12) Patent Application Publication
(10) Pub. No.: US 2007/0281349 A1
(43) Pub. Date: Dec. 6, 2007

(54) Industrial bioreactor and method of use in continuous protein and lipid recovery system

(75) Inventor: Jacek Jaczynski, Morgantown, WV (US)

Correspondence Address: WEST VIRGINIA UNIVERSITY RESEARCH CORPORATION, 886 CHESTNUT RIDGE ROAD, P.O. BOX 6216, MORGANTOWN, WV, 26506-6216, US

(73) Assignee: West Virginia University

(21) Appl. No.: 11/447,352

(22) Filed: Jun. 6, 2006

(51) Int. Cl. C12M 1/36

(52) U.S. Cl. 435/286.5; 435/289.1

(57) ABSTRACT

The present invention provides for an industrial scale bioreactor and for a use of the industrial scale bioreactor in a continuous protein and lipid recovery system. The industrial scale bioreactor comprises a pH-resistant container able to hold at least 150 gallons wherein said pH-resistant container further comprises, a means to add influent to said pH-resistant container, a means to remove effluent from said pH-resistant container, one or move thermocouples able to measure the temperature within the pH-resistant container, a mixer reversibly attached within said pH-resistant container, and a means to monitor and control the pH within said pH-resistant container. The continuous protein and lipid recovery system comprises a homogenizer, a means to connect said homogenizer to a first bioreactor wherein said first bioreactor is maintained at a programmed pH level away from the isoelectric point of the protein so it is water-soluble, a separator, a means to connect said first bioreactor to said separator, a second bioreactor maintained at a programmed pH level at the isoelectric point of the protein so it is water-insoluble, a means to connect said separator to said second bioreactor, a second separator, a means to connect said second separator to said second bioreactor, and a means to monitor and control the temperature throughout the protein and lipid recovery process.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

1. Field of Invention

This invention relates to an industrial scale bioreactor and for a use of the industrial scale bioreactor in a continuous protein and lipid recovery system.

2. Description of the Prior Art

The transformation of raw materials into foods inevitably generates some type of by-products and the processing of aquatic foods is no exception. Therefore, developing new technologies for the full utilization of these by-products is of critical importance to the future economic viability of this industry. In traditional and non-industrialized fisheries, where most of the labor is provided by personnel with skills often passed down by generations, the fish is almost completely utilized for human consumption, animal feed, or as plant fertilizer. The economy-driven industrialization of fisheries brought incredible advances, but at the same time, the amounts of by-products generated during harvesting and processing increased dramatically. Typical examples are commercial shrimp trawling, krill processing and mechanized fish filleting. In shrimp trawling, sometimes 90% of the total catch volume corresponds to species with no commercial value and this by-catch is therefore most often discarded.

The meat recovery yield during commercial processing of whole krill (Euphausia superba) is extremely low, fluctuating between 10 and 15% by weight. Finally, when fish are mechanically processed for fillets, the recovery yields are typically 30-40% of fillets and the by-products account for 60-70% by weight of the whole fish. While it is not uncommon to just grind-and-discard this 60-70% of fish by-products, this practice should be considered an irresponsible utilization of natural resources and should be used instead to fulfil human nutritional needs.

About 100 million metric tons of the global fisheries production is processed for direct human consumption. Commercial processing of fish such as cod, salmon, trout, tilapia, seabream and pollack typically yields about 30-40% of fillets as products, and while meat and oil left on the remaining by-products range widely, they account typically for 20-30 and 5-15% of whole fish weight, respectively. The 100 million metric tons processed for direct human consumption do not include the fish by-catch and discards estimated to account for an additional 30 million tons of available catch not yet utilized for human consumption.

Another aquatic resource that is scarcely utilized for human consumption is krill. At present, krill is commercially utilized mostly by the reduction fisheries to manufacture fish feed. The development of an appropriate technology to efficiently convert this resource into food could contribute significantly to fulfil nutritional needs for proteins and help alleviate over-fishing and stock depletion problems affecting several aquatic species. This vast resource has been estimated at 400-1550 million metric tons with a sustainable annual harvest of about 70-200 million metric tons. The krill biomass potentially available for human food is comparable to that of all of the other aquatic species currently under commercial exploitation and is probably the largest of any multi-cellular animal species on the planet. Krill, small crustaceans that resemble shrimp, are not fully utilized for human consumption due to the lack of efficient meat recovery technology. Krill meat is literally liquefied at high rates by extremely active proteolytic enzymes released during harvest.

The biomass of aquatic by-products and underutilized species is staggering. At the same time, over-fishing, stock depletion, and other environmental issues associated with aquatic food production are increasingly more important. Also, the world population is increasing and it is becoming more difficult to meet nutritional needs for proteins and lipids from aquatic resources.

A number of U.S. patents have addressed the issue of the recovery of animal, namely fish, protein via isoelectric points. These patents include U.S. Pat. Nos. 6,005,073; 6,136,959; 6,288,216; and 6,451,975. All of the processes take advantage of the low protein solubility at their isoelectric point. None of the above patents, however, disclose or teach the novel features of the present invention as disclosed herein. The drawback to all of the above procedures is that they cannot be applied outside of a laboratory without significant changes required for efficient industrial production. They are also less efficient due to the batch recovery process utilized rather than the continuous protein and lipid recovery system disclosed herein. The upscale needed for an industrial setting has not been practical before the present invention. No current bioreactor exists with the ability to allow a user to control the level of pH and agitation while monitoring temperature in a large-scale, continuous environment. In addition, industrial scale bioreactors of about 150 to about 300 gallons will result in a flow rate of about 15 to 30 gallons per minute, respectively. This flow rate will also allow processing capability at 25,000 or 50,000 lbs of input material per day, respectively. The flow rate will also allow the required 10 minute reaction time needed for a statistical particle entering a bioreactor to reach either solubility or precipitation. The design of these bioreactors will allow modular connections of many bioreactors in parallel if more processing capability is desired for various operations. The continuous protein recovery system also allows for continuous protein pH adjustment in each bioreactor. A batch system requires pH adjustment in steps and significantly increases the viscosity of the solution between about pH 8-10 and pH 3.5-4.5 impeding good mixing and resulting in a pH gradient. Therefore, the batch system can cause some processing issues including incomplete or inefficient protein separation, foaming, etc.

The present invention relates to an industrial scale bioreactor comprising a pH-resistant container able to hold at least about 150 gallons, a means to add influent, a means to remove effluent, one or more thermocouples, a mixer, and a means to monitor and control the pH within said pH-resistant container. The invention further describes the use of the bioreactor within a continuous protein recovery system comprising a homogenizer, a means to connect said homogenizer to a first bioreactor wherein said first bioreactor is maintained at a programmed pH level, a separator, a means to connect said first bioreactor to said separator, a second bioreactor maintained at a programmed pH level, a means to connect said separator to said second bioreactor, a second separator, a means to connect said second separator to said second bioreactor, and a means to monitor and control the temperature throughout the protein recovery process.

### BRIEF SUMMARY OF THE INVENTION

The present invention provides for an industrial scale bioreactor comprising a pH-resistant container able to hold at least about 150 gallons, a means to add influent, a means to remove effluent, one or more thermocouples, a mixer, and a means to monitor and control the pH within said pH-resistant container. The industrial bioreactor will allow for continuous input and output of material to and from the bioreactor in a system allowing for the control of pH, amount of agitation desired by a user, and monitoring of temperature. The industrial bioreactor can be used in a continual protein recovery, however, the use is not limited to such as it can be used in any setting in which a large amount of a sample needs to be kept at a constant pH level, and/or agitated.

It is an object of the present invention to allow the large scale, continuous control of fluid sample pH. One of the benefits of the continuous system is that the protein exposure to the pH is short and therefore protein degradation is lessened. The pH gradients formed in batch processes are also eliminated during continuous control.

Another object of the present invention is a continuous protein recovery system comprising a homogenizer, a means to connect said homogenizer to a first bioreactor wherein said first bioreactor is maintained at a programmed pH level, a separator, a means to connect said first bioreactor to said separator, a second bioreactor maintained at a programmed pH level, a means to connect said separator to said second bioreactor, a second separator, a means to connect said second separator to said second bioreactor and a means to monitor and control the temperature throughout the protein recovery process. The large continuous protein recovery system allows for a more efficient recovery than a batch system with a more consistent final product.

The present invention further provides for both the bioreactor and continuous protein recovery system to have a means of temperature monitoring throughout the process. The bioreactor includes one or more thermocouples. The continuous protein recovery system may be placed entirely within a temperature controlled environment and the entire system may be monitored by thermocouples. The continuous protein recovery system further details that the temperature controlled room should be kept at about 40° F. for optimal protein recovery.

Anther aspect of the present invention is that the bioreactor is comprised of a pH-resistant material preferably high density polyethylene to decrease the cost required to maintain an industrial scale operation.

The present invention further details an ability to control the flow rate in and out of the bioreactors. The input rate can be controlled by an influent pump set at a constant rate and the effluent may be removed by either a pump or preferably an overflow opening.

Another aspect of the present invention is the ability to control the rate of agitation and therefore the amount of air that taking into the solution within the bioreactor. The invention will have a mixer which preferably will have at least one mixing baffle.

The present invention will also have the ability to add additional materials to the industrial scale bioreactor such as emulsion breakers and flocculants. These materials may be added in the continuous protein recovery system in either the first or second industrial scale bioreactor or both.

### BRIEF DESCRIPTION OF THE SEVERAL VIEWS OF THE DRAWING

These drawing are for illustrative purposes only and are not drawn to scale.

FIG. 1 is frontal view of an embodiment of an industrial scale bioreactor.

FIG. 2 is a general process diagram of the use of the industrial scale bioreactor in an example of a continuous protein recovery system. The first pH adjustment in the illustrative example is away from the protein isoelectric point to cause the protein to become water-soluble. The pH adjustment can be either above 11 or below 3.5 for proteins with an isoelectric point of about 5.5.

### DETAILED DESCRIPTION OF THE INVENTION

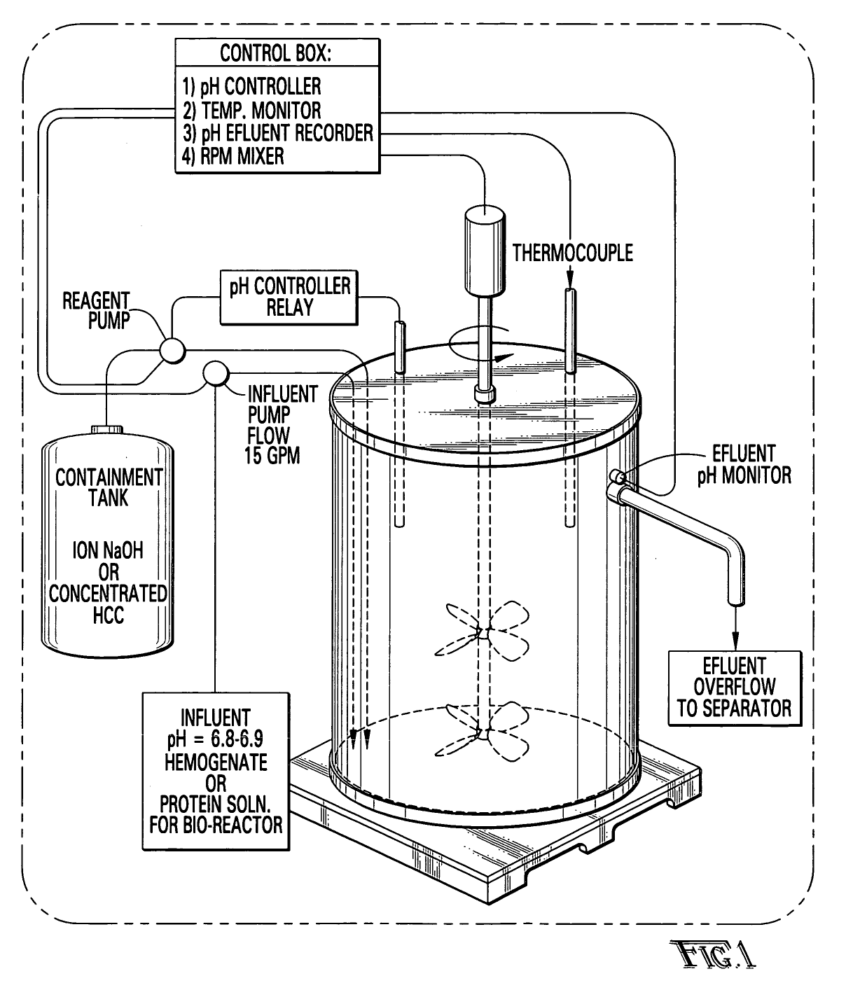

The present invention is an industrial scale bioreactor and the use of the industrial scale bioreactor in a continuous protein recovery system. The preferred embodiment of the industrial scale bioreactor is illustrated in FIG. 1. The industrial scale bioreactor comprises a pH-resistant container of industrial scale size able to hold at least 150 gallons. The pH-resistant container is preferably comprised of heavy duty high density polyethylene to decrease the cost significantly while maintaining the required industrial strength and pH-resistance. The shape of the pH-resistant container is cylindrical to facilitate mixing to reduce any pH gradients and prevent air uptake thereby minimizing excessive foaming. A pH-resistant container of between 150 to 300 gallons will result in a flow rate at about 15 to 30 gallons per minute, respectively. The pH-resistant container may also be skid-mounted for portability and safety.

The pH-resistant container is further comprised by a means to add influent to the container. The influent can be any solution that the user desires for use in the industrial scale bioreactor. The means to add influent can be any means with a pressure gradient of either positive pressure at the desired influent or a negative pressure at an influent outlet or both pressures creating a gradient. The pressure gradient may be created by a pump which can be at either the influent source or the outlet or any other area to create a pressure gradient, the pressure gradient can be caused by a gravity feed, the pressure gradient could be created by adding pressurized gas from the influent source to a pressurized pH-resistant container, or the pressure gradient can be created by any means standard within the art. In addition, the pH-resistant container includes a means to remove the effluent. The means to remove the effluent is also a pressure gradient. The preferred means to remove effluent is by an overflow opening on the side of the pH-resistant container to reduce cost and simplify the system, but the means to remove effluent could also be a pump, a pressurized bioreactor, or any other means of creating a pressure gradient that would be readily apparent to one skilled in the art.

The industrial scale bioreactor will also contain one or more thermocouples to monitor the temperature within the pH resistant container. The thermocouple is a temperature sensor that monitors the temperature of the contents of the pH-resistant container. The thermocouples can be mounted in the wall of the pH-resistant container so that the thermocouple is able to monitor the temperature of the contents or the thermocouples may be added into the pH-resistant container through either a space in a lid covering the pH-resistant container through the top of an uncovered container. Multiple thermocouples may be utilized at various depths in order to gauge the temperature of the contents of the pH-resistant container at various levels. The industrial scale bioreactor may also include a means to change the temperature within the pH-resistant container. The means to change the temperature can be any method readily apparent to one skilled in the art.

The industrial scale bioreactor also has a mixer reversibly attached within the pH-resistant container, preferably attached through the opening of a removable lid. The mixer can be of any standard mixing method, however, the preferred mixer has at least one mixing baffle. The use of two mixing baffles on the mixer is preferred, because it will prevent air uptake by the solution within the pH-resistant container.

The industrial scale bioreactor is also comprised of a means to monitor and control the pH within the pH-resistant container. The means to monitor the pH can be any conventional pH monitor such as a pH meter and the means of pH control can be any conventional way to add any acid or base into the pH-resistant container. The preferred means to monitor and control the pH within the pH-resistant container comprises a pH controller relay and pH chart recorder. The pH controller relay is connected to tanks of either acid or base or both acid and base. The pH within the pH-resistant container is constantly monitored and can be recorded by the pH chart recorder at desired intervals. The pH controller relay is programmed to maintain the pH at a desired pH by adding either acid or base to the solution within the pH-resistant container.

The industrial scale bioreactor may also contain a means to add any additional material the user desires. The means to add additional material is a conveyer for solids or a pump for gases, liquids, and mixtures of any phases. The conveyer can be any standard conveyer for solids such as a conveyer belt or a screw conveyer while any standard pump can be used by one skilled in the art to transport gases, liquids, or mixtures of any phases. The additional material can be anything a user desires in any phase of matter. The additional material could be a reactant, a product, a catalyst, an additive, an inhibitor, or any material desired by the user for any purpose.

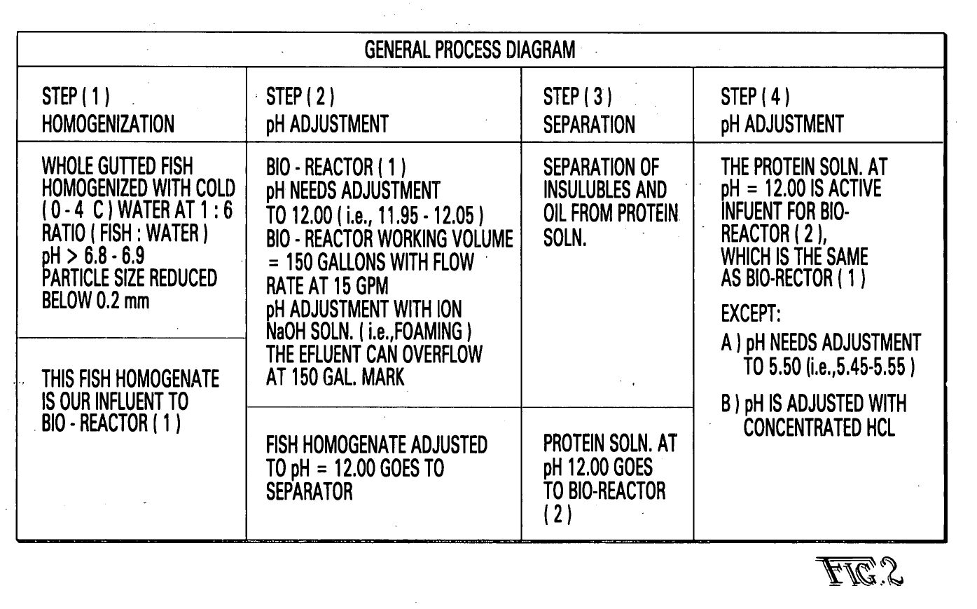

The continuous protein recovery system comprises a homogenizer, a means to connect said homogenizer to a first bioreactor wherein said first bioreactor is maintained at a programmed pH level away from the isoelectric point of the protein so it is water-soluble, a separator, a means to connect said first bioreactor to said separator, a second bioreactor maintained at a programmed pH level at the isoelectric point of the protein so it is water-insoluble, a means to connect said separator to said second bioreactor, a second separator, a means to connect said second separator to said second bioreactor, and a means to monitor and control the temperature throughout the protein recovery process. An embodiment of the continuous protein and lipid recovery system is shown in FIG. 2.

The first step in the continuous protein recovery system is homogenization. Any standard homogenizer and any protein source may be used. Some examples of successful sources are fish by-products, whole fish, and krill but any source of protein that is able to be homogenized to about 0.2 mm or less can be used. The source/water product is called homogenate after homogenization with water. The homogenate is then loaded into the first bioreactor, which is the same as described above, through a means of connection with the preferred method being an influent pump although the means of connection could be any that is readily apparent to one skilled in the art.

The first bioreactor is kept at a constant, monitored pH away from the protein isoelectric point. The isoelectric point is a pH at which proteins have the net electrostatic charge equal to zero causing the proteins to precipitate. As the proteins diverge from their isoelectric point due to changes in pH, the protein water-solubility is increased. The preferred proteins, fish proteins, typically have an isoelectric point of 5.5. The pH can be maintained at either a higher or lower level for the proteins to become water-soluble. The preferred levels are either a pH above 11.00 or below 3.50. However, a pH above 13.00 or below 1.50 can cause protein degradation. The pH of the first bioreactor is maintained away from the isoelectric point so that the proteins are water-soluble.

The homogenate is then removed from the first bioreactor to a separator. The means of connecting the first bioreactor to the separator can be changed by one skilled in the art, however, the preferred means of connecting the first bioreactor to a separator is by either a pump or an overflow opening. The separator can be any separation device, for example an industrial-scale decanter-centrifuge, which would be readily apparent to one skilled in the art. The separator will separate the insoluble material and the oil from the remaining protein solution. The remaining protein solution is then removed from the separator and added to a second bioreactor through a means of connection with the preferable method being an influent pump. The second bioreactor is kept at a monitored pH level at the isoelectric point. Therefore, the water-soluble protein becomes water-insoluble and able to be removed from the protein solution through any conventional separation method in a second separator, for example an industrial-scale decanter-centrifuge. The second bioreactor is connected to the second separator through a means of connection which can be any conventional type of connection that allows the protein solution to flow from the second bioreactor to the second separator although the preferred method is either an overflow opening or a pump. All of the continuous protein recovery equipment will work in a cold environment. The entire process is subject to a means to monitor and control the temperature throughout the protein recovery process. In order to minimize muscle protein and lipid degradation the temperature should be kept at or below about 40° F. The temperature control can be achieved by placing the entire continuous protein recovery system inside a temperature controlled room such as a walk-in cooler.

In addition, both bioreactors in the continuous protein and lipid recovery system could have additives added to either the homogenate or protein solution, respectively through a means to add additional material which is located on the preferred embodiment of the pH-resistant container. Emulsion breakers and flocculants are commonly used in the food industry. Emulsion breakers act at the protein-lipid interface hindering interactions, and therefore, contribute to the destabilization of the protein-lipid emulsion. Continual addition of emulsion breakers in the first bioreactor will aid subsequent separation of phospholipids which are the major cause of rancidity or the fishy smell in fish muscle tissue. Flocculants interact with proteins by non-specific bonds, and therefore, significantly increase the particle size of the protein molecules. According to the Stoke's law, by increasing the particle size by a factor of 3, the separation velocity under the g force increases by a factor of 9. A second separation is often inefficient, because the solubilized and precipitated proteins are very small. However, the food-grade flocculants efficiently increase the size of the protein particles during the continuous protein recovery and make a final separation more efficient.

What is claimed is:

1. An industrial scale bioreactor comprising:
a pH-resistant container able to hold at least 150 gallons wherein said pH-resistant container further comprises;
a means to add influent to said pH-resistant container;
a means to remove effluent from said pH-resistant container;
one or more thermocouples able to measure the temperature of contents within said pH-resistant container;
a mixer able to agitate contents within said pH-resistant container; and
a means to monitor and control the pH within said pH-resistant container.

2. The industrial scale bioreactor of claim 1 wherein said pH-resistant container is high density polyethylene.

3. The industrial scale bioreactor of claim 1 wherein said pH-resistant container is cylindrical in shape.

4. The industrial scale bioreactor of claim 1 wherein said means to add influent is a pressure gradient.

5. The industrial scale bioreactor of claim 4 wherein said pressure gradient is a pump.

6. The industrial scale bioreactor of claim 1 further comprising a skid mounting of said pH-resistant container.

7. The industrial scale bioreactor of claim 1 further comprising a means to change the temperature within said pH-resistant container.

8. The industrial scale bioreactor of claim 1 further comprising a means to add additional material to said pH-resistant container.

9. The industrial scale bioreactor of claim 8 wherein said means to add additional material is a conveyer for solids or a pump for gases, liquids, and mixtures of any phases.

10. The industrial scale bioreactor of claim 1 wherein said means to remove effluent is a pressure gradient.

11. The industrial scale bioreactor of claim 1 wherein said pressure gradient is an overflow opening.

12. The industrial scale bioreactor of claim 11 wherein said pressure gradient is a pump.

13. The industrial scale bioreactor of claim 1 wherein said means to remove effluent further comprises a pH monitor.

14. The industrial scale bioreactor of claim 1 wherein said mixer comprises at least one mixing baffle.

15. The industrial scale bioreactor of claim 1 wherein said means to control the pH is a pump attached to containment tanks containing acid and base, respectively.

16. The industrial scale bioreactor of claim 1 wherein said means to monitor and control the pH is a pH controller relay attached to pH monitors wherein said pH controller relay is attached to a reagent pump attached to containment tanks containing either an acid or a base.

17. The industrial scale bioreactor of claim 1 further comprising a lid removably attached to said pH-resistant container.

18. The industrial scale bioreactor of claim 17 wherein said lid has openings for said means to monitor and control the pH, said mixer, and said thermocouple to pass through.

19. A continuous protein recovery system comprising:
a homogenizer with the ability to mix the homogenized particles with liquid to create homogenate;
a means to connect said homogenizer to a first bioreactor so that a homogenate can be added to the first bioreactor through the means of connection wherein said first bioreactor comprises a pH-resistant container able to hold at least 150 gallons wherein said pH-resistant container further comprises an influent pump attached to said pH-resistant container, a means to remove effluent from said pH-resistant container, one or more thermocouples able to measure the temperature of contents within said pH-resistant container, a mixer able to agitate contents within said pH-resistant container, and a means to monitor and control the pH within said pH-resistant container at a programmed pH level away from the isoelectric point of a protein so that said protein is water-soluble;
a first separator;
a means to connect said first bioreactor to said first separator so that the homogenate can be removed from the first bioreactor to the separator through the means of connection;
a second bioreactor comprising a pH-resistant container able to hold at least 150 gallons wherein said pH-resistant container further comprises an influent pump attached to said pH-resistant container, a means to remove effluent from said pH-resistant container, one or more thermocouples able to measure the temperature of contents within said pH-resistant container, a mixer able to agitate contents within said pH-resistant container, and a means to monitor and control the pH within said pH-resistant container at a programmed pH level away from the isoelectric point of a protein so that said protein is water-soluble;
a means to connect said first separator to said second bioreactor so that the remaining protein solution can be added to the second bioreactor from the first separator through the means of connection;
a second separator connected to the second bioreactor by a means to connect said second separator to said second bioreactor so that the remaining protein solution can be removed from the second bioreactor through the means of connection; and
a means to monitor and control the temperature of the continuous protein recovery system.

20. The continuous protein recovery system of claim 19 wherein said means to connect said homogenizer to a first bioreactor is a pump.

21. The continuous protein recovery system of claim 19 wherein said means to connect said first pump with said separator is by an overflow opening.

22. The continuous protein recovery system of claim 19 wherein said means to connect said separator to said second bioreactor is by a pump.

23. The continuous protein recovery system of claim 19 wherein said first and second bioreactors are maintained at a programmed pH level by a pH control comprising a pH monitor, an acid solution, a basic solution, and a pump connecting said acid solution and said basic solution to said bioreactors.

24. The continuous protein recovery system of claim 19 wherein said means to monitor the temperature of the continuous protein recovery system is a thermocouple.

25. The continuous protein recovery system of claim 19 wherein said means to control the temperature of the continuous protein recovery system is by performing the process in a temperature controlled room.

26. The continuous protein recovery system of claim 19 further comprising the addition of emulsion breakers and flocculants to said first and second bioreactors.
